(19) United States
(12) Patent Application Publication
(10) Pub. No.: US 2024/0225769 A1
(43) Pub. Date: Jul. 11, 2024

(54) A CONTAINER

(71) Applicant: Multigate Medical Products Pty Ltd, Villawood (AU)

(72) Inventors: Amy LEEDHAM, Villawood (AU); Joshua MCCARTHY, Villawood (AU); Caitlin NEIL, Villawood (AU); Abdu MOUKAHAL, Villawood (AU); Stephen LA, Villawood (AU)

(21) Appl. No.: 18/291,507

(22) PCT Filed: Jul. 21, 2022
(86) PCT No.: PCT/AU2022/050777

§ 371 Date: Jan. 23, 2024

(30) Foreign Application Priority Data
Jul. 21, 2021 (AU) 2021902243

(51) Int. Cl. A61B 50/30; A61B 50/20; A61B 90/00

(52) U.S. Cl. CPC A61B 50/3001; A61B 50/20; A61B 90/08; A61B 2050/21; A61B 2050/3002; A61B 2050/3008; A61B 2090/0804

(57) ABSTRACT

A container (10) for holding a primary sharp object, the container (10) including a cushioning layer (20), the cushioning layer (20) having a pre-formed indentation (25), the indentation (25) being configured to receive and hold the primary sharp object therein.

### TECHNICAL FIELD

The present invention relates to a container. In particular, the present invention relates to a container for receiving, holding and/or storing sharp objects. According to a further example, the present invention relates to a needle or sharps counter and a blade remover, provided separately or together in a container.

### BACKGROUND OF THE INVENTION

The following references to and descriptions of prior proposals or products are not intended to be and are not to be construed as, statements or admissions of common general knowledge in the art. In particular, the following prior art discussion does not relate to what is commonly or well known by the person skilled in the art, but assists in the understanding of the inventive step of the present invention of which the identification of pertinent prior art proposals is but one part.

Containers such as needle counters are typically used by a scrub nurse during a surgical procedure to count and organise sharps such as sutures or the like. At the end of the procedure, the needle counter can be closed and placed directly into a sharps bin. However, needles are often not placed in the container in a manner which allows safe handling.

Further, during surgical procedures, nurses must often also remove blades from scalpel handles by positioning the blade pointing away from the body, grasping the blade with an instrument such as a needle holder or artery forceps, then pull the blade downwards and away from the body releasing it from the handle.

The current methods for removing and then storing sharp objects can be harmful to medical practitioners as there is a risk of needle-stick injury due to the manual handling of the sharp objects and objects not being placed in a sharps container appropriately. Further, there is a risk that if not counted properly, sharp objects may be inadvertently left within a patient during surgery, which can have often fatal consequences.

The present invention seeks to provide a container which may ameliorate the foregoing shortcomings and disadvantages or which will at least provide a useful alternative.

### SUMMARY OF THE INVENTION

According to one aspect of the invention, there is provided herein a container or needle counter for holding a primary sharp object, the container including a cushioning layer, the cushioning layer having a pre-formed indentation, the indentation being configured to receive and hold the primary sharp object therein.

According to one example, the pre-formed indentation includes a marker at or near the indentation.

According to yet another example, the marker is any one or a combination of: alpha-numerical marker; and, a mark at least partially surrounding the indentation.

In one example, the marker is a border surrounding the indentation.

In a further example, the alpha-numerical marker is a number part of a group of sequential numbers to allow for counting of the number of primary sharp objects held within the container.

According to yet another example, the container has a plurality of pre-formed indentations, each of the plurality of pre-formed indentations being formed across the width of the cushioning layer.

In one form, the container has a plurality of pre-formed indentations formed along a length of the container.

In yet another example, the plurality of pre-formed indentations are spaced equidistantly from each other along the width and the length of the cushioning layer.

In one form, there is provided in an open configuration, an interior of the container has a first side including the cushioning layer and a second side having a magnetic layer.

According to another form, the magnetic layer has one or more ridges or undulations configured to magnetically hold a secondary sharp object at an angle.

In another example, the container includes a blade remover, wherein a scalpel or knife placed within the blade remover safely removes a blade from the scalpel or knife.

In one further example the primary sharp object can be any one or a combination of: one or more sutures; one or more blades; and, one or more needles.

According to another aspect, there is provided herein a container for holding a sharp object, the container including a magnetic layer, the magnetic layer having one or more undulations, wherein the one or more undulations are configured to magnetically hold the sharp object at an angle.

According to one example, the magnetic layer is formed on an inner surface of the container.

In another aspect, there is provided herein a container for holding a sharp object, the container including a blade remover, wherein a scalpel or knife placed within the blade remover safely removes a blade from the scalpel or knife.

In another aspect, there is provided herein a blade remover for removing a blade from a scalpel or knife, wherein when the scalpel or knife is placed within the blade remover safely removes a blade from the scalpel or knife.

According to one example, the blade remover includes a prong configured to catch the blade of the scalpel or knife to thereby release the blade from the scalpel or knife.

In yet another example, the blade remover includes a posterior surface; an outwardly facing surface formed by a body of the prong; and, a cavity formed between the posterior surface and the outwardly facing surface; wherein the prong has a resilient hooked leg extending from the body of the prong towards the posterior surface and biased towards the posterior surface, such that when the scalpel is received within the cavity, the resilient hooked leg bends to allow the blade to pass through the cavity, and catches the blade such that movement back through the cavity is limited to allow releasing of the blade from the scalpel.

In a further example, the blade remover includes a first prong and a second prong, the first prong and second prong being spaced apart from each other and forming a channel there between.

In another example, each of the first prong and the second prong have a respective first hooked leg and a second hooked leg.

In yet another example, one of the first hooked leg and second looked leg is shorter than the other.

According to a further example, the first and second prongs allow for the blade to pass through the cavity by allowing the blade to pass through a space formed between the first and second hooked legs and the posterior surface.

In another example, once the blade has passed the first and second hooked legs, the first and second hook legs snap back towards the posterior surface.

According to a further example, a handle of the scalpel or knife is pulled though the channel such that the blade is caught by the first and second hooked legs and is released/dislodged from the handle of the scalpel or knife.

According to one example, the container includes a clip configured to connect or attach the container to a surface.

According to another example, the surface is a surgical table.

It will be appreciated that any combination of features, elements and examples as described herein is possible.

### BRIEF DESCRIPTION OF THE DRAWINGS

The invention may be better understood from the following non-limiting description of a preferred embodiment, in which:

FIG. 1A is a schematic showing a perspective view of an example embodiment of a container as described herein;

FIG. 1B is a schematic showing a top view of the example container of FIG. 1A;

FIG. 1C is a schematic showing a side view of the example container of FIG. 1A;

FIG. 1D is a schematic showing a top view of the example container of FIG. 1A;

FIG. 1E is a schematic showing a cross-sectional view A-A of the container in FIG. 1D;

FIGS. 2A and 2B are photographic images showing an example of a container as described herein;

FIG. 3 is a schematic showing an example of a cushioning member as described herein;

FIGS. 4A and 4B are photographic images showing an example of a container as described herein;

FIG. 5A is a schematic showing a perspective view of an example of a blade remover as described herein;

FIG. 5B is a schematic showing a perspective view of the example blade remover of FIG. 5A, cut in half;

FIG. 6A is a schematic showing a perspective view of an example of a blade remover as described herein;

FIG. 6B is a schematic showing a perspective view of the example blade remover of FIG. 6A, cut in half;

FIG. 6C is a schematic showing a top view of the example blade remover of FIG. 6A;

FIG. 6D is schematic showing a front view of the example blade remover of FIG. 6A;

FIG. 6E is a schematic showing a side view of the example blade remover of FIG. 6A;

FIG. 7A is a schematic showing a side view of an example of a blade remover;

FIG. 7B is a schematic showing a front perspective view of the example blade remover of FIG. 7A;

FIGS. 7C, 7E, 7G, 7I, 7K are schematics showing a side view of the blade remover in use;

FIGS. 7D, 7F, 7H, 7J, 7L are schematics showing a front perspective view of the blade remover in use;

FIG. 8 is a photographic image showing another example of a blade remover formed as part of a container;

FIGS. 9A and 9B are schematics of another example of a container having a blade remover in the top portion of the container, in closed configuration; and,

FIG. 10 is a schematic of the container of FIGS. 9A and 9B, in open configuration.

### DETAILED DESCRIPTION OF THE DRAWINGS

An example of a container, such as a container for storing sharp objects, 10, is shown in the Figures and described in more detail below.

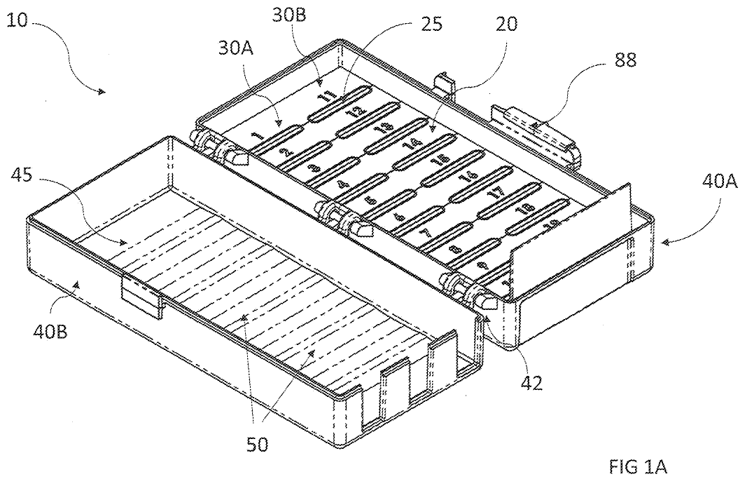

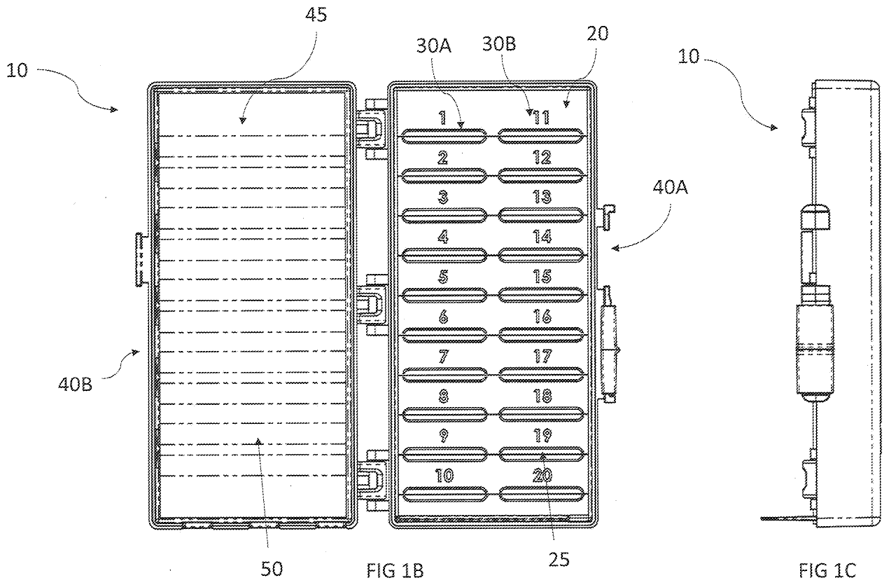

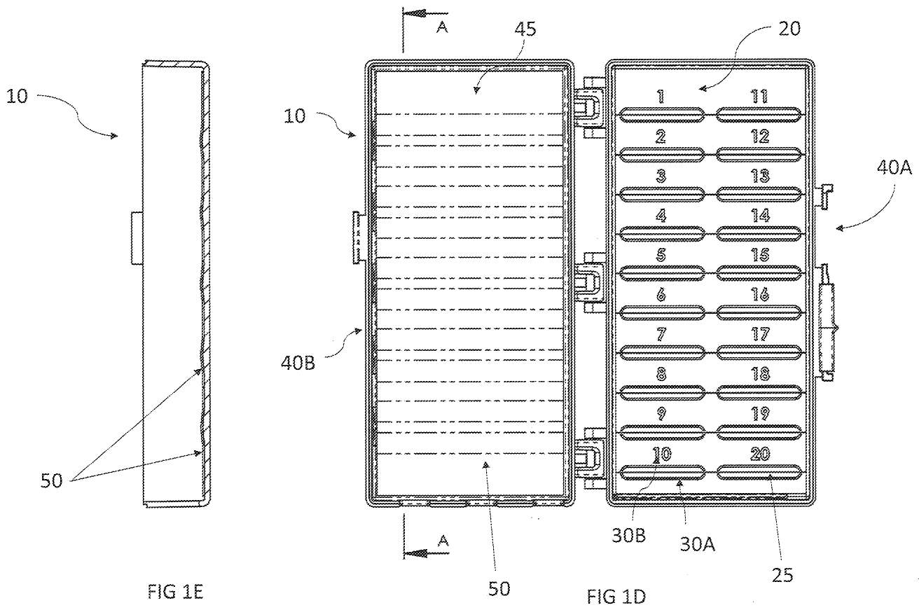

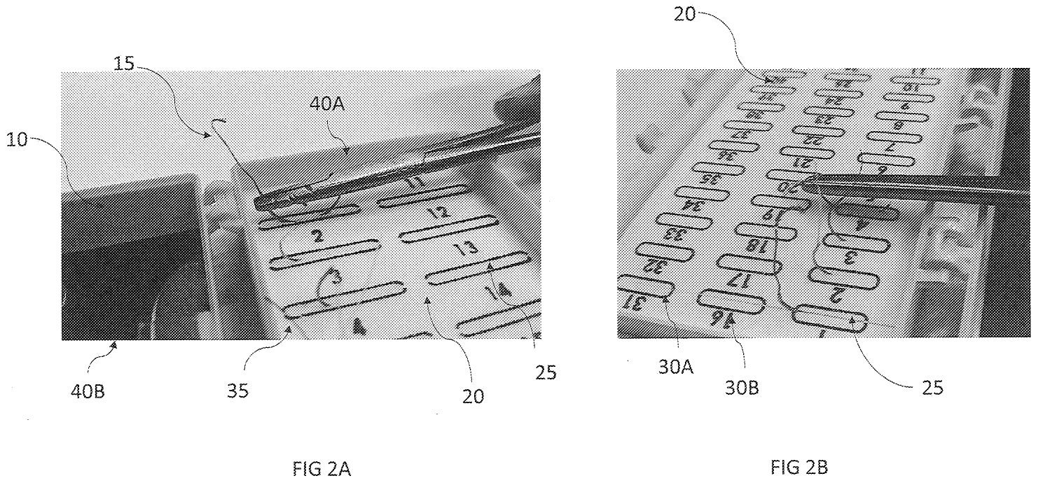

According to a particular example, FIG. 1A to FIG. 3 show examples of a container 10 for holding (storing or receiving) a primary sharp object 15. As an example, the primary sharp object can be a needle, a suture, and/or a blade. In this particular example, the container 10 includes a cushioning layer 20, such as, for example, a foam layer or the like.

Typically, the cushioning layer 20 has a slit or indentation 25, which can be pre-formed in the cushioning layer 20 such that the indentation 25 is configured to receive and hold the primary sharp object 15 therein.

According to one example, the indentation 25 can include a marker, indicator or identifier 30A or 30B at or near the indentation. Thus, for example, the marker 30A/30B can be any one or a combination of an alpha-numerical marker and/or a mark which at least partially surrounds the indentation 25 which can be used to visually highlight where the indentation is in/on the cushioning layer 20. It will be appreciated that the markers 30A/30B can make the indentation 25 more visually identifiable and thus easier to see where the slit/indentation is in the cushioning layer 20 to thereby place a specific sharp object 15 at least partially into the indentation 25.

It will be appreciated that any form of markers (typically visual) can be used. In the examples shown herein, an alpha-numeric marker near one or a portion of a slit can be used so that a medical practitioner can identify the number of sharp objects that have been placed within the cushioning layer 20. This can assist in surgical procedures in determining that all sutures, for example, are counted for. Notably, in the examples shown, sequential numbers, such as numbers 1 to 20, are provided at or near the indentations to allow for counting of the number of sharp objections held within the container, but it will be appreciated that any indicator can be used to count the number of sutures or sharps stored therein.

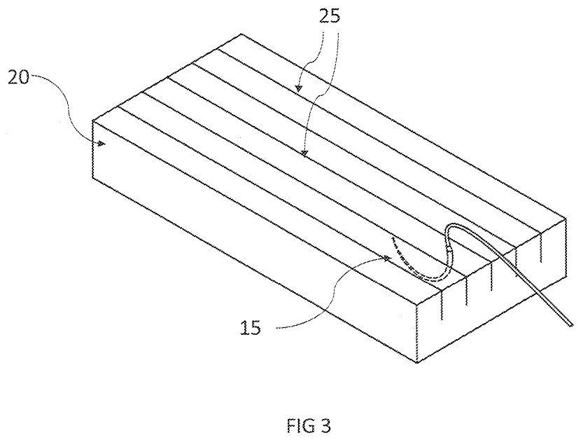

Further, the marker can be a border that can surround the indentation either entirely or partially, to further assist in locating the sharps object into the cushioning layer. As shown in the examples of FIG. 3, the cushioning layer can have a plurality of pre-formed indentations. In these examples, each of the plurality of pre-formed indentations are formed across the width of the cushioning layer. They can also be formed along the length of the cushioning layer/container. Further, and as discussed below, the indentations can be spaced equidistantly from each other along the width and the length of the cushioning layer, which can allow for the sharp objects stored therein to be spaced apart from each other in a manner which allows the objects to be easily seen and picked up for use and/or counted for accordingly.

Thus, the container 10 can be a needle-counter for holding/storing one or more sharp objects such as sutures, needles, or blades.

According to one particular example, the cushioning layer 20 is a cushioning member which can be formed of a piece, sheet, block, layer of foam or the like, like a foam block, which can then be attached to the inside of the container 10 and has one or more or a series of slits or indentations 25 cut into it. In a particular example, if there are a plurality of slits 25, they can be formed in the cushioning or foam layer 20 at regular intervals and extend from one side of the foam 20 to the other. As described above, it will be appreciated that the one or more slits 25 can run along the cushioning member 20 either along its length, or along its width, and can even be placed diagonally. Furthermore, the one or more slits 25 can be provided as discrete slits or connected along the foam length/width, such as shown in FIG. 3. According to another example, the slits can also be offset from the edges of the foam (such as for example, 5 mm from the edge) or can run all the way to the edges of the foam member 20.

Although in the examples shown, the slits 25 are placed at regular intervals to each other, it will be appreciated that the length of the intervals can differ between different sized needle counters/containers. For example, one foam block can have slits spaced 10-12 mm (such as 11.2 mm) apart and another can have the splits 9-8 mm (such as 9.4 mm apart). Furthermore, the size of the container 10 can determine the size of the foam block which can be placed inside it, which can then determine the number of slits within the layer 20. Typically, the slits need to be far enough apart to allow space for numbers/graphics to be printed between them, but close enough to make efficient use of the space on the foam. As an example, the splits could be provided at a range of 2 mm-20 mm for slits at regular intervals. However, it will be appreciated that the foam may only have 1 slit or a couple of splits interspersed anywhere along the foam or slits irregularly distributed.

In a further example, the slits 25 do not typically cut all the way through the foam 20.

It will be appreciated that the use of the foam slits or indentations 25 can allow for hands-free suture or sharps management (as an example of a sharp object 15) in a surgical environment. As an example, a scrub nurse can manoeuvre the suture 15 within the foam 20 before removing the suture 15 so there isn't a need to reposition the suture 15 with their fingers before handing the suture to the surgeon, which can reduce the risk of sharps injury. The provision of one or more slits 25 within the cushioning member 20 can allow for the suture to be positioned within the foam such that when removed, it is in the appropriate position for a handover and does not need to be further handled. Furthermore, the foam slits 25 can allow the sutures 15 to be pushed into the foam 20 without blunting the tip of the suture 15.

According to one specific example, when the container 10 is in an open configuration, as shown for example in FIGS. 1A, 1B, 1D, 2A, 2B, 4B, and FIG. 10, an interior 35 of the container 10 has a first side 40A and a second side 40B. In these examples, the first side 40A includes the cushioning layer 20 and the second side 40B has a magnetic layer 45, which will be further described below.

Typically, the first side 40A and the second side 40B are opposing sides formed across a hinge member 42 such that when the container 10 is in a closed configuration, as shown for example in FIGS. 1A and 1B, the first side 40A and the second side 40B face each other across a connection such as a hinge assembly or the like. Thus, the container 10 can be hingeably openable to retrieve any sharp object 15 (or 65) and closable to store and/or hold sharp objects accordingly. Notably, it will be appreciated by persons skilled in the art that the cushioning layer 20 and the magnetic layer 45 can be formed on the same side within the container and not necessarily on opposing sides 40A, 40B. Thus for example, the cushioning layer and/or magnetic layer can be formed in a base of the container 10 such as the side 40A and the side 40B can act as a lid to close the container 10 and maintain one or more sharp objects therein.

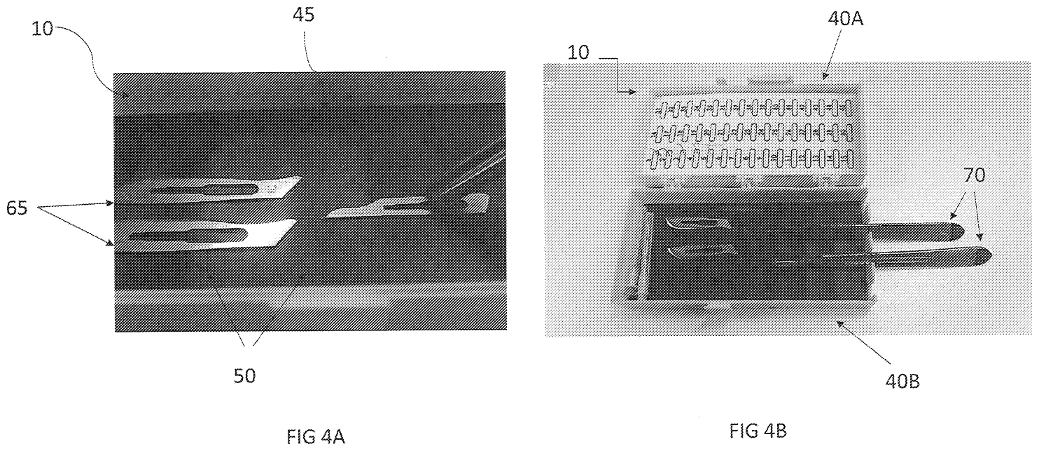

Now turning to the magnetic layer 45, as shown particularly in FIGS. 4A and 4B as well as FIGS. 1A to 1E, the magnetic layer 45 can be a magnetic strip or sheet formed within the container 10.

In one example, the magnetic layer 45 can be formed such that the magnetic layer 45 has one or more ridges or undulations 50 configured to magnetically hold a secondary sharp object 65 at an angle.

It will be appreciated that one or more ridges 50 can be formed either within (that is integrally with) the magnetic layer 45 or on the second side 40B such that when a magnetic sheet 45 is attached to the inside of the container 10 at the side 40B, the magnet sheet 45 conforms to the shape of these ridges 50, thereby providing an undulating surface on top of the magnetic sheet 45.

Accordingly, it will be appreciated by persons skilled in the art that the ridges 50 on the magnet surface 45 lift a sharp object 65 such as a blade or the like at least partially away from the surface of the magnetic strip 45 thus making it easier and quicker to pick up scalpel blades 65 given they are at least partially lifted off or spaced apart from the magnetic layer 45.

It will be appreciated that, depending on the size of the container 10, the ridges 50 can be spaced at regular intervals, such as for example, 18 mm to 21 mm apart. The number of ridges typically depends on however many can fit in the container 10 or on the magnetic layer 45 whilst being spaced apart at regular intervals. It will be appreciated that although a plurality of ridges 50 are shown, it is also possible to have only one or a couple interspersed at regular intervals on the magnetic layer 45.

In the examples shown, the container 10 has a rectangular profile, although it will be appreciated that any other shape of container is possible. Further, as shown particularly in FIG. 3, in an open configuration, the container 10 has the magnetic layer 45 on one internal surface and the cushioning layer 20 on the other opposing internal surface. In these examples, the magnetic layer 45 and the cushioning layer 20 fit within the internal surfaces of the container 10 entirely along the internal surface width and length, although it will be appreciated that they can be formed to be shorter in width and/or length of the internal surface of the container 10.

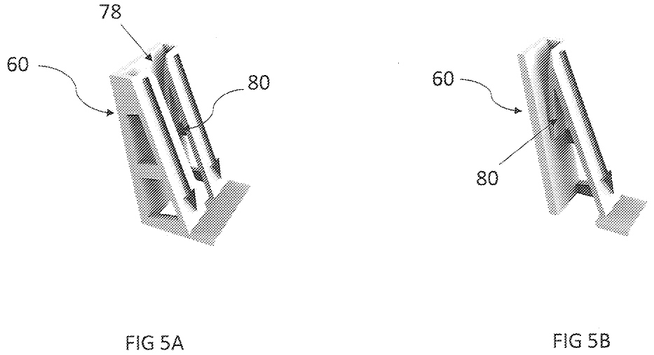

Thus, the container 10 can be a closable case with a combination of foam and magnetised surfaces inside for placing sutures and scalpel blades therein. Thus, the container 10 can be a combined blade-remover and needle counter. Alternatively, the container 10 can solely be a needle counter or a blade-remover. In yet a further example, the container 10 can include a blade remover 60. Examples of the blade remover 60 is shown in FIGS. 5A to 10.

The blade remover 60 can allow for safe removal of a blade 65 from a scalpel or knife 70 placed within the blade remover 60. The blade remover 60 typically includes a prong 80 which is configured to catch the blade 65 of the scalpel or knife 70 to thereby release the blade 65 from the scalpel or knife 70.

Typically, as a user inserts a scalpel 70 into the blade remover 60, a cavity 78 formed in the blade remover 60, receives the blade 65 and a portion of a handle 68 of the scalpel 70. The blade remover 60 has one or more or a set of prongs 80 formed within the blade remover 60, which flex inwardly to allow the blade 65 and handle 68 to pass inside/within the blade remover 60. When the user pulls the handle 68 back, the prongs 80 catch the blade 65 and the blade 65 is thus pulled off the handle 68.

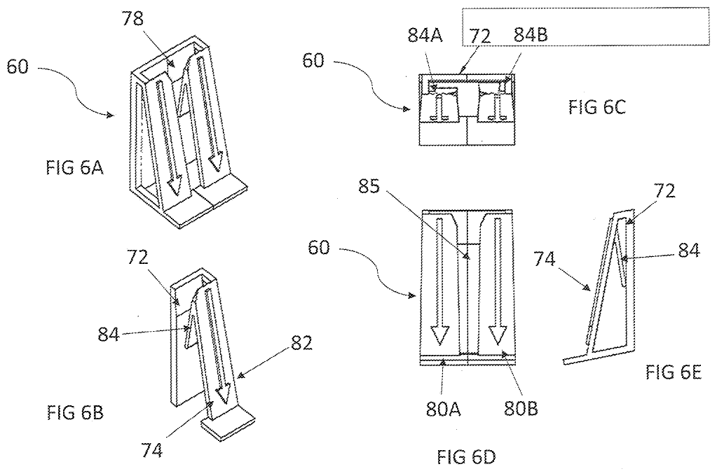

Referring more specifically to FIGS. 6A to 6E, according to one example, the blade remover 60 includes a posterior surface 72, an outwardly facing surface 74 formed by a body 82 of the prong 80, and the cavity 78 is formed between the posterior surface 72 and the outwardly facing surface 74.

In these examples, the prong 80 typically has a resilient hooked leg 84 extending from the body 82 of the prong 80 towards the posterior surface 72 and biased towards the posterior surface 72, such that when a scalpel or knife 70 is received within the cavity 78, the resilient hooked leg 84 bends to allow the blade 65 to pass through the cavity 78, and catches the blade 65 such that movement back through the cavity 78 is limited to allow releasing of the blade 65 from the scalpel handle 68.

It will be appreciated that the blade remover 60 can include any number of prongs 80—that is one or more prongs or a plurality/set of prongs 80. In one particular example, the blade remover 60 includes a first prong 80A and a second prong 80B, where the first prong 80A and second prong 80B are typically spaced apart from each other and form a channel 85 there between.

Thus, each of the first prong 80A and the second prong 80B can have a respective first hooked leg 84A and a second hooked leg 84B. Typically, the first and second prongs 80A, 80B allow for the blade 65 to pass through the cavity 78 by allowing the blade 65 to pass through a space formed between the first and second hooked legs 84A, 84B and the posterior surface 72, when the blade 65 is pushed there between. Accordingly once the blade 65 has passed the first and second hooked legs 84A, 84B, the first and second hook legs 84A, 84B snap back towards the posterior surface 72. The handle 68 of the scalpel or knife 70 is then typically pulled though the channel 85 such that the blade 65 is caught by the first and second hooked legs 84A, 84B and is released/dislodged from the handle 68 of the scalpel or knife 70.

According to one particular example, one of the first hooked leg 84A and second looked leg 84B can be shorter than the other. Thus for example, as shown particularly in FIG. 6C, when the outwardly facing surface 74 is facing a user, the hooked leg 84A of the left prong 80A is typically shorter than the hooked leg 84B of the right prong 80B. In this particular example, the prongs 80 are formed such that they correspond with the shape of the blade 65. Thus for example, the blades 65 typically have a slanted edge opposite the pointy end of the blade 65. When using the blade remover 60, the scalpel is typically inserted with the sharp side of the scalpel facing towards the left, which means the taller end of the slanted edge is on the left hand-side of the blade remover. Thus, having a shorter left prong means the two prongs align with the shape of this slanted edges of the blade 65 better, and can allow for better removal of the blade 65 from the scalpel 70. However, it will be appreciated that the blade remover 60 can still work if the prongs 80 were the same length or formed to associate with a different shaped blade 65.

Notably, FIGS. 6A to 6D also show that the outwardly facing surface 74 can include indicators such as arrows which can assist communicating to a medical professional that for this particular blade remover 60, the scalpel 70 is to be pushed into the blade remover 60 in the direction of the arrows.

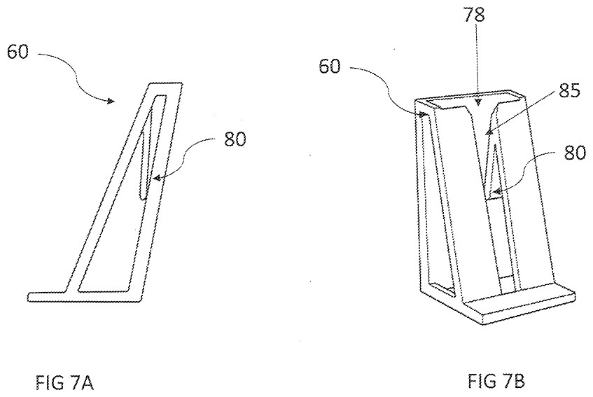

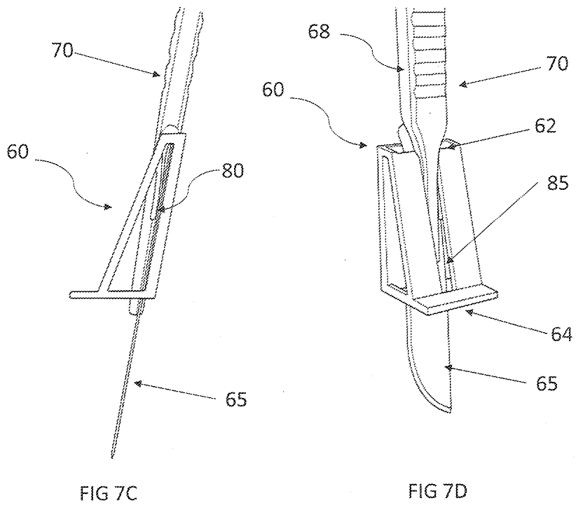

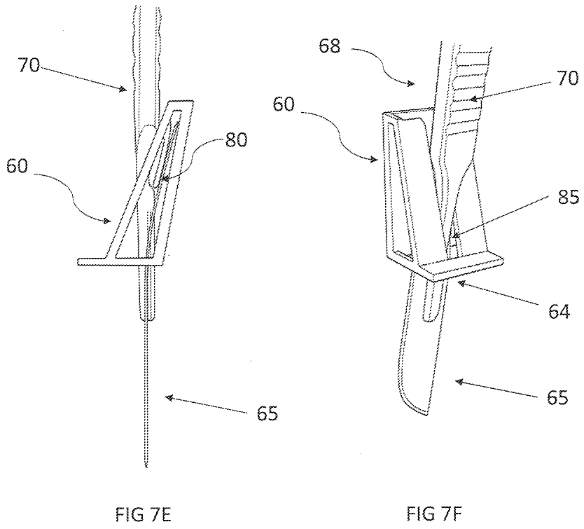

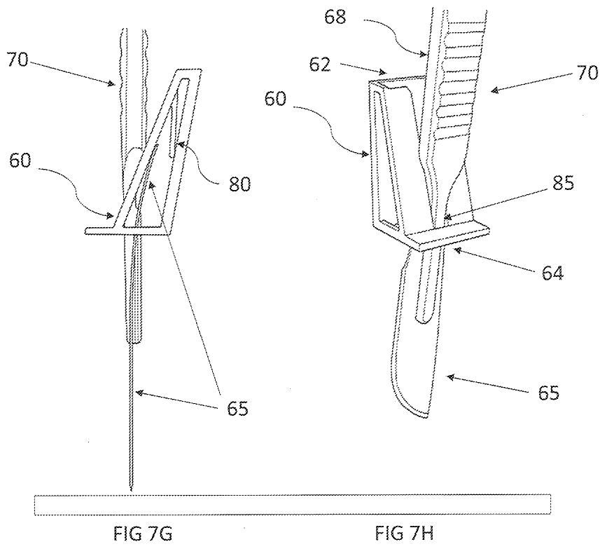

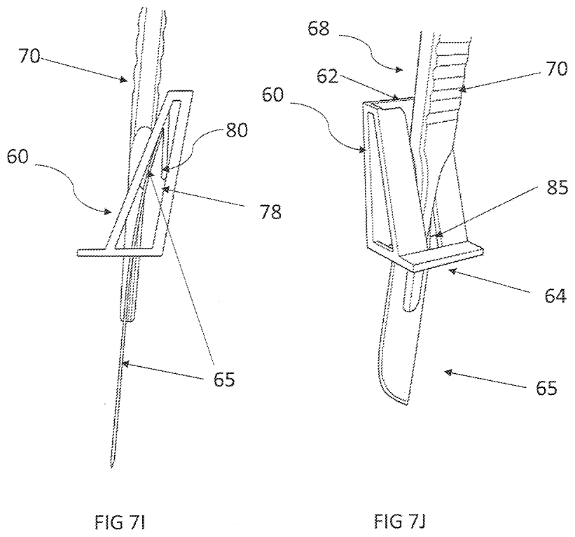

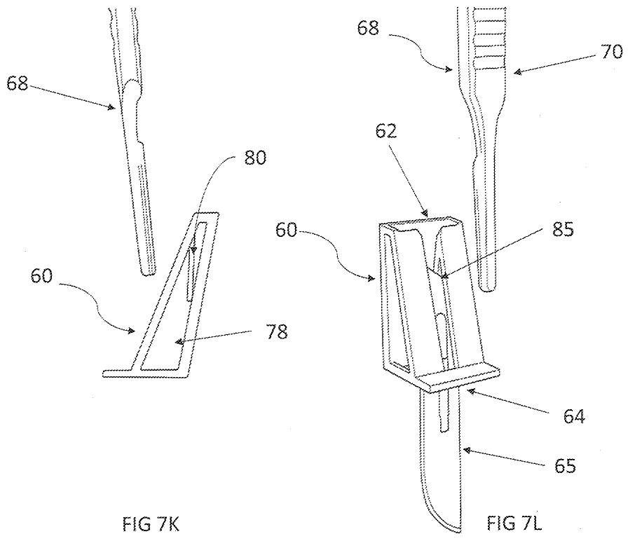

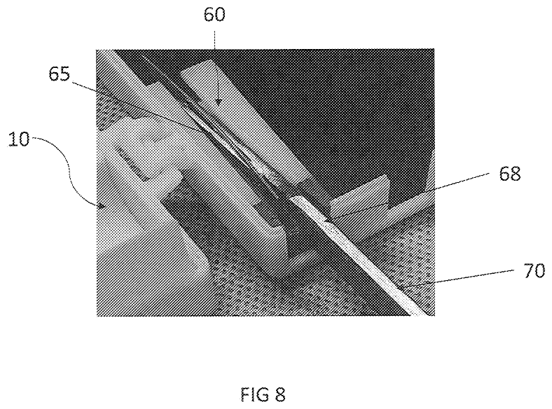

FIGS. 7C to 7L show further example steps that a user may go through to remove the blade 65 from a scalpel or knife 70.

At step 1, as shown for example in FIGS. 7C and 7D, the scalpel 70 is inserted downwards into the blade remover 60 until a thicker section of the handle 68 contacts a receiving end 62, which can be a top portion of the blade remover 60. Typically, the flexible prongs or hooks 80 are deflected forward as the blade 65 of the scalpel 70 passes behind them.

At step 2, as shown for example in FIGS. 7E and 7F, the scalpel handle 68 is tilted forward, which can allow a narrow section of the handle 68 to pass through the central channel 85 in the blade remover 60. Meanwhile the blade 65 is constrained inside the blade remover 60 which causes it to flex and partially separate from the handle 68. The flexible prongs 80 are deflected further by the blade 65.

At step 3, as shown for example in FIGS. 7G and 7H, the scalpel handle 68 is pushed further downwards which causes the blade 65 to pass beyond the flexible prongs 80. This creates an audible sound as the prongs snap back into their original position. Simultaneously, the wider section of the handle 68 reaches the bottom or ejecting end 64 of the central channel 85 of the blade remover 60. This can also provide visual confirmation that it is now okay to pull the handle back upwards and through the receiving end 62.

At step 4, as shown for Example in FIGS. 71 and 7J, the scalpel handle 68 is pulled upwards. The blade 65 is trapped within the cavity 78 of the blade remover 60 by the flexible prongs 80. Similar to a one way valve, the blades 65 can go in one direction but they can't come back out the receiving end 62 of the blade remover 60.

At step 5, as shown for example in FIGS. 7K and 7L, once the scalpel handle 68 is pulled upwards and out of the blade remover 60, the blade 65 separates completely from the handle 68 and falls through the cavity 78 and onto a surface below the blade remover 60 (notably, the surface is not shown in this example). The handle 68 of the scalpel 70 is then removed from the top 62 of the blade remover 60 accordingly.

It will be appreciated that the blade remover 60 utilises a push-pull operation of scalpel blade removal which can be performed by one hand. Further advantages of the blade remover 60 include but are not limited to:
Providing a visual confirmation of when the blade is removed—when the wider part of the handle reaches the bottom of the blade remover channel, the user can pull the handle back
Providing an audible click when the blade is removed—when the blade passes beyond the prongs, they snap back into their original position which produces a sound
Can be used many times for the removal of multiple blades. Notably, the container can also be opened to allow for retrieval or re-use of blades
Providing a treacherous path through which blades cannot exit the container after they're removed and thus blades are safely contained

Accordingly, it will be appreciated that the container 10 can allow for the safe removal of a scalpel blade with a single-handed operation, can indicate that the used scalpel blade has been removed by an audible sound and/or a visual cue, and can allow for the safe containment of the scalpel blade immediately after the scalpel blade is removed. Notably, the user does not need to move blades into the needle counter after they're removed—they're automatically deposited onto the internal magnet surface which when the container is in the closed configuration, is formed underneath the blade remover 60 (thereby acting as a chute) which when the blade 65 is displaced, the blade 65 will typically drop onto the magnetic surface underneath).

It will be appreciated that the blade remover can form a part of the container either separately to the cushioning layer 20 and the magnetic layer 45 described herein, or they can form a part of the same container 10.

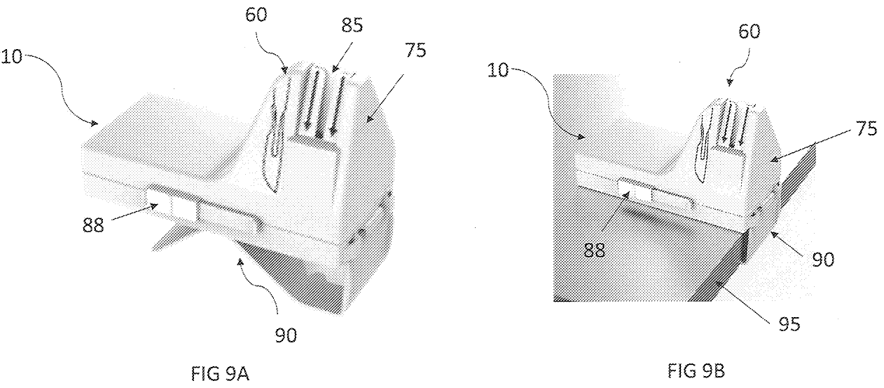

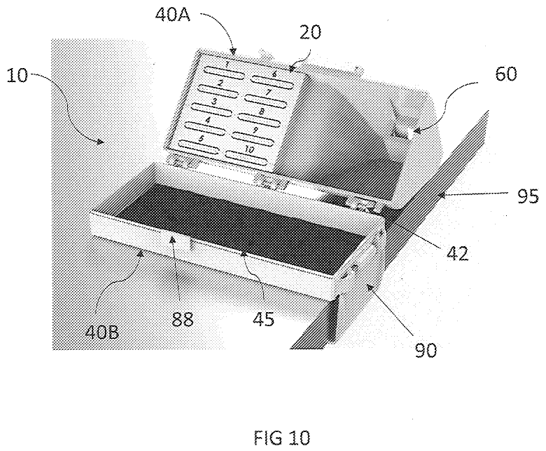

As shown particularly in FIGS. 9A, 9B and FIG. 10, the blade remover 60 in these examples is formed or integrated into a top or top portion 75 of the container 10. However, it will be appreciated the blade remover 60 can also be formed within one of the sides 40A/40B of the container 10 as shown in FIG. 8.

FIGS. 8 to 10 show examples of the container 10 having the dual purpose of being a blade remover as well as a needle counter. In these example, when the two sides 40A and 40B are closed, they form a large cavity where blades and sutures can be placed/held.

The container 10 can also have a clasp 88 which can allow the container 10 to be opened and locked shut. Notably, it will be appreciated that any features described herein can be provided in combination or separately. Thus for example, the needle counter can include either one or both of the cushioning member and magnetic layer as described herein and can be provided together with or separate from the blade remover. Thus, the blade remover can be provided separately to the needle counter as its own device.

According to a further example, the container 10 described herein can be sterile and provided/used in a sterile field in a surgical procedure. This can allow reuse of blades and scalpel handles as they can remain in the sterile field in a surgical procedure. As shown particularly in FIGS. 9A to 10, a clip 90 on a bottom surface or base of the container 10 can attach the container 10 to a table 95 such as a medical/surgical table or a back table in a surgical procedure. The clip 90 secures the container 10 to the table 90 stabilising the container 10 such that scalpel blades can be removed using a single hand.

Notably, the clip 90 allows the container 10 to be secured to a surface, such as for example, the table 95 even if the table is covered, and further, the container 10 can be secured to the table 95 without breaking sterility that is, the container 10 can be slid via the clip 90 onto the table 95 without a medical practitioner needing to touch below the horizontal surface of the table, thereby maintaining sterility.

Thus, the container described herein can allow for effective sharps management during a surgical setting through the use of one container. As an example, the container 10 can minimise the risk of needle-stick or sharp injuries and can further assist surgical staff in ensuring that the number of sharp objects are accurately counted for as they are safely removed and stored within the container 10.

The term “comprise” and variants of that term such as “comprises” or “comprising” are used herein to denote the inclusion of a stated integer or integers but not to exclude any other integer or integers, unless in the context or usage an exclusive interpretation of the term is required.

Those skilled in the art will appreciate that the invention described herein is susceptible to variations and modifications other than those specifically described. All such variations and modifications are to be considered within the scope and spirit of the present invention the nature of which is to be determined from the foregoing description.

1. A container for holding a one or more sharp objects, an interior side of the container having a magnetic layer, wherein the magnetic layer has one or more ridges or undulations configured to magnetically hold the respective one or more sharp object at an angle, wherein the container further includes a cushioning layer, the cushioning layer having a pre-formed indentation, the indentation being configured to receive and hold another sharp object therein.

2. The container of claim 1, wherein the pre-formed indentation includes a marker at or near the indentation.

3. The container of claim 2, wherein the marker is any one or a combination of:
a. alpha-numerical marker; and,
b. a mark at least partially surrounding the indentation.

4. The container of claim 3, wherein the mark is a border surrounding the indentation.

5. The container of claim 3, wherein the alpha-numerical marker is a number part of a group of sequential numbers to allow for counting of the number of sharp objects held within the container.

6. The container of claim 1, wherein the cushioning layer has a plurality of pre-formed indentations, each of the plurality of pre-formed indentations being formed across the width of the cushioning layer.

7. The container of claim 1, wherein the cushioning layer has a plurality of pre-formed indentations formed along a length of the container.

8. The container of claim 6, wherein the plurality of pre-formed indentations are spaced equidistantly from each other along the width and the length of the cushioning layer.

9. The container of claim 1, wherein in an open configuration, an interior of the container has a first side including the cushioning layer and a second side including the magnetic layer.

10. The container of claim 1, wherein the container includes a blade remover, wherein a scalpel or knife placed within the blade remover safely removes a blade from the scalpel or knife.

11. The container of claim 1, wherein the sharp object can be any one or a combination of one or more sutures; one or more blades; and, one or more needles.

12. The container of claim 1, wherein the container includes a clip configured to connect or attach the container to a surface.

13. The container of claim 12, wherein the surface is a surgical table.
